(19) United States
(12) Patent Application Publication
(10) Pub. No.: US 2005/0261619 A1
(43) Pub. Date: Nov. 24, 2005

(54) Vortex-flow air removal in a blood perfusion system

(75) Inventor: Eric L. Gay, Ann Arbor, MI (US)

Correspondence Address: TERUMO CARDIOVASCULAR SYSTEMS CORPORATION, 6200 JACKSON ROAD, ANN ARBOR, MI, 48103, US

(73) Assignee: Terumo Cardiovascular Systems Corporation

(21) Appl. No.: 11/136,047

(22) Filed: May 24, 2005

Related U.S. Application Data
(60) Provisional application No. 60/573,923, filed on May 24, 2004.

(51) Int. Cl. A61M 37/00

(52) U.S. Cl. 604/4.01

(57) ABSTRACT

An air removal system removes air from blood flowing in a perfusion system. A first elongated, substantially cylindrical chamber is provided having a central longitudinal axis between an upstream end and a downstream end, wherein the chamber provides a substantially unobstructed blood flow path therethrough along a wall of the chamber, and wherein the downstream end provides an air-reduced blood flow to the perfusion system. A vortex flow introducer is located substantially at the upstream end. A bubble stop is provided which blocks the central longitudinal axis prior to the downstream end. An air removal line is coupled to the central longitudinal axis between the upstream end and the bubble stop. An air extraction unit has an input coupled to the air removal line, an air output for coupling to a vacuum source via a valve, and a blood output. A blood return line is coupled to the blood output for returning blood passing through the air extraction unit to a return point that is downstream of the bubble stop.

### CROSS REFERENCE TO RELATED APPLICATIONS

This application claims priority to co-pending U.S. provisional application Ser. No. 60/573,923, filed May 24, 2004.

### STATEMENT REGARDING FEDERALLY SPONSORED RESEARCH

Not Applicable.

### BACKGROUND OF THE INVENTION

The present invention relates in general to extracorporeal blood perfusion systems, and, more specifically, to an air removal system and device for separating entrained air from blood flowing in the system.

When heart surgery is performed ‘on pump’, steps are taken to remove air entrained in the blood flowing in the extracorporeal blood circuit. Preferably, air removal occurs upstream of the pump. Typically, either a cardiotomy reservoir with defoamer or a flexible venous reservoir (FVR) has been employed. An FVR typically comprises a sealed bag with a luer valve or stopcock at its upper end for manually removing excess air. A cardiotomy reservoir comprises a hard shell for collecting and storing blood which is then supplied to the pumped system. The collection chamber is open to atmosphere and the blood is at atmospheric pressure. Any air bubbles in the blood rise to the top of the collection chamber. Blood is resident in the reservoir for a time that is sufficiently long for air to separate. A blood defoamer is often mounted in the reservoir to aid in the breakdown of foam bubbles in the chamber. Substantially bubble-free blood is drawn out of the reservoir at the bottom. The cardiotomy reservoir can also be used for filtration of particulates or for addition of fluids or pharmacological agents.

Blood from a patient can be collected passively or actively. Passive drainage is accomplished by catheterizing the patient, connecting the catheter with tubing to a cardiotomy or FVR, and siphoning the blood into the cardiotomy or FVR. Active drainage is accomplished by using either a pump or vacuum source on the drainage line to pump or suction blood from the access site. The resulting blood flow rate is greater than what is obtained using passive drainage. When drainage is passive, the pressure in the extracorporeal circuit upstream of the blood pump typically becomes slightly positive relative to atmospheric. When drainage is active, the pressure in the circuit upstream of the pump frequently becomes less than atmospheric. Either a cardiotomy or FVR may be used when drainage is passive. An FVR will not work during active drainage because the negative pressure in the circuit will cause the FVR to collapse.

Certain advantages could be realized by eliminating the use of the cardiotomy reservoir. For instance, a reduction in blood contacting surface areas, a reduction of blood to air interface, a reduction of fluid priming volume of the perfusion circuit, and elimination or reduction of the amount of blood-to-defoamer contact are all expected to improve patient outcome. Since an FVR provides a closed system (i.e., not open to atmosphere) it can achieve some of these advantages to a certain degree, but it cannot be used when active drainage is desired because of the tendency to collapse under negative pressure.

Hard shell reservoirs have been used in a closed configuration in order to implement vacuum-assisted blood collection from the patient (i.e., systems known as VAVD for Vacuum Assisted Venous Drainage). The large reservoirs generate a large blood to air interface and often use defoamer in the flow path to prevent air bubbles leaving the reservoir. In a VAVD reservoir, the blood path is continuously connected to and at the same pressure as the vacuum source. They require monitoring by the perfusionist to maintain a stable level in the reservoir by balancing blood inflow and outflow. Also known are kinetic-assist devices using a smaller chamber wherein suction for collecting blood from the patient is directly obtained from a blood pump. However, these systems require an active electronic sensor such as an ultrasonic sensor for detecting the presence of collected air and an electronically-controlled purge valve that is triggered when air is sensed. Cost and potential reliability issues associated with active sensing and purging are disadvantageous. It would be advantageous to be able to remove significant quantities of air from blood flowing at high flow rates in a passive manner (i.e., without either electronic sensors or requiring a balancing of inflow and outflow rates) and doing so whether the pressure within the system is higher or lower than atmospheric pressure.

### SUMMARY OF THE INVENTION

The present invention provides an air removal device and method with low prime volume, efficient air removal, and minimal exposure of blood to a defoamer. The device described herein does not collapse under negative pressure and can be used in place of a cardiotomy reservoir for both passive and active drainage procedures.

In one aspect of the invention, an air removal system is provided for removing air from blood flowing in a perfusion system. A first elongated, substantially cylindrical chamber is provided having a central longitudinal axis between an upstream end and a downstream end, wherein the chamber provides a substantially unobstructed blood flow path therethrough along a wall of the chamber, and wherein the downstream end provides an air-reduced blood flow to the perfusion system. A vortex flow introducer is located substantially at the upstream end. A bubble stop is provided which blocks the central longitudinal axis prior to the downstream end. An air removal line is coupled to the central longitudinal axis between the upstream end and the bubble stop. An air extraction unit has an input coupled to the air removal line, an air output for coupling to a vacuum source via a valve, and a blood output. A blood return line is coupled to the blood output for returning blood passing through the air extraction unit to a return point that is downstream of the bubble stop.

### BRIEF DESCRIPTION OF THE DRAWINGS

FIG. 1 is a diagrammatic view of a perfusion system of the present invention.

FIG. 2 is a cross-sectional perspective view of a voraxial air separation device according to a first embodiment.

FIG. 3 is a cross section showing an alternative bubble stop.

FIG. 4 is a block diagram showing a system of the invention using multiple stages of voraxial air separation devices.

FIG. 5 is a block diagram of a multi-stage system using a shared air extraction device.

FIG. 6 is a partial cross section of a voraxial air separation device used as a first stage in the system of FIG. 5.

FIG. 7 is a partial cross section of voraxial air separators and air extraction unit according to the system of FIG. 5.

FIG. 8 is a vertical cross section of an integrated multiple stage voraxial air separation device.

FIG. 9 is a horizontal cross section of the voraxial air separation device taken along line 9-9 of FIG. 8.

FIG. 10 is a vertical cross section of an alternative embodiment of the voraxial air separation device having a recursive loop.

FIG. 11 is a top plan view of the device of FIG. 10.

FIG. 12 is a perspective view of another alternative embodiment of the air separation device.

FIG. 13 is a vertical cross section along line 13-13 of FIG. 12.

### DETAILED DESCRIPTION OF PREFERRED EMBODIMENTS

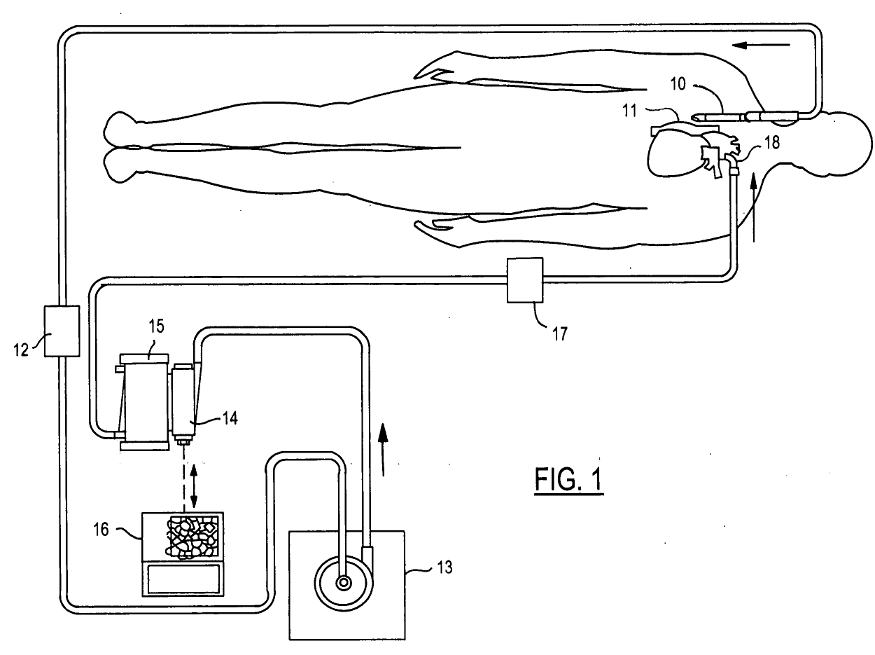

FIG. 1 shows a simplified diagram of a perfusion system for supporting on-pump coronary artery bypass graft surgery. A venous catheter 10 is inserted at 11 into the right side of a patient's heart or the superior or inferior vena cava. Venous blood flow is driven by an arterial pump 13 which may be comprised of a centrifugal pump, for example. Blood passes through a heat exchanger 14 and then to an oxygenator 15. A blood heater/cooler 16 is connected to heat exchanger at 14 for selectably heating or cooling blood as is required during different phases of surgery. Oxygenated blood is conducted to an arterial cannula 18 to return the oxygenated blood to the patient's aorta.

Air in the form of a bolus or bubbles can be introduced into the blood at the point of extraction from the body due to a leak around the venous catheter, for example. It is desirable to remove entrained air prior to the blood entering the pump and oxygenator. Thus, an air removal device 12 is preferably inserted into the venous line. Rather than or in addition to air removal device 12, an air removal device 17 may be used in the arterial side of the circuit.

Among other objectives, the present invention seeks to minimize prime volume of the perfusion circuit as well as reducing surface area of blood contact and the exposure of blood to air (the air/blood interface) or to defoamers. It is further desirable to handle large volumes of both air and blood while removing large amounts of air in a short period of time while using a device that does not collapse when the circuit pressure is below atmospheric pressure.

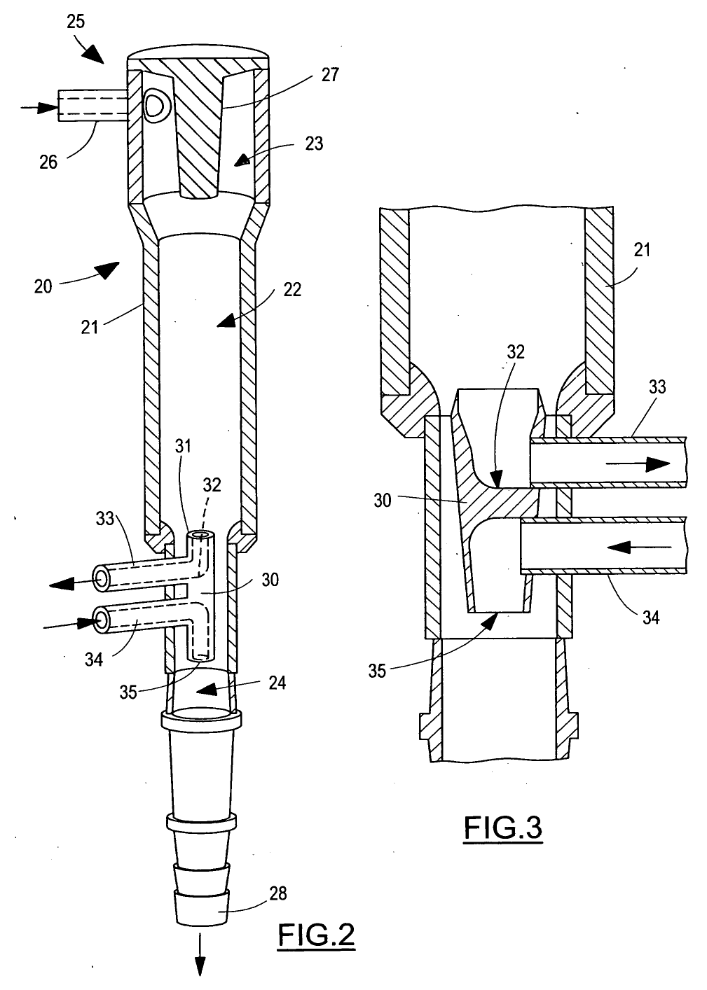

In accordance with the foregoing objectives, an air separation device as shown in FIG. 2 is utilized. Voraxial device 20 creates an axial vortex of the blood/air mixture in order to segregate air from the blood using a centrifugal flow wherein denser blood moves to the outer radial portion of the chamber and less dense air moves to the central longitudinal axis. Thus, a housing 21 defines an elongated, substantially cylindrical chamber 22 along a central longitudinal axis between an upstream end 23 and a downstream end 24. The blood/air mixture enters the upstream end 23 via a vortex flow introducer 25 which may comprise a tangentially/oriented inlet 26. Vortex flow introducer 25 may also include a central obstruction 27 having a downward-facing conical wall disposed around the central longitudinal axis. A substantially unobstructed blood flow path is created along the wall of chamber 22 so that the segregated blood flows to downstream end 24 and out through an outlet 28 providing an air-reduced blood flow to the perfusion system. The radial diameter of chamber 22 may be generally narrowing from upstream end 23 to downstream end 24 in order to maintain the strength of the vortex flow.

A diverter body 30 is mounted in chamber 22 between upstream end 23 and downstream end 24 for completely blocking the central longitudinal axis. Thus, air bubbles collecting along the central longitudinal axis are blocked from reaching outlet 28. The blocked areas include a bubble stop surface 31 together with a bubble stop blocking wall 32 inside diverter 30. Surface 31 and wall 32 are substantially perpendicular to the central longitudinal axis and cooperatively block and collect air bubbles. Air along the central longitudinal axis is picked-off by diverter 30 and flows through an air removal line 33 to an air extraction unit (not shown). As shown in FIG. 2, the pick-off point and bubble stop may be integrally formed into an elbow.

Depending upon the flow rate into device 20 and the concentration of air within the mixture, a greater or lesser amount of air and blood may be flowing in air removal line 33. Since a significant amount of blood may be present in air removal line 33, it must be recovered for return to the patient. The air extraction unit is shown in greater detail below and preferably comprises a valved air separation device as shown in co-pending application Ser. No. 11/118,726, filed Apr. 29, 2005, entitled “Air Removal Device With Float Valve For Blood Perfusion System”, incorporated herein by reference.

A returning blood flow from the air extraction unit is provided to a blood return line 34 and to a return point 35 that is downstream of the bubble stop. Preferably, return point 35 is on the central longitudinal axis because it is at a low pressure and because that provides the least disruption of the main blood flow through device 20. Since the majority of the blood flowing through device 20 does not pass through the air extraction unit, it avoids contact with defoamers. Furthermore, the air extraction unit can be made more compact in size since a lowered volume of blood passes through it, which further reduces the priming volume of the system as a whole.

FIG. 3 shows an alternative embodiment of diverter 30 wherein bubble stop surface 32 provides substantially all of the blocking of the central longitudinal axis.

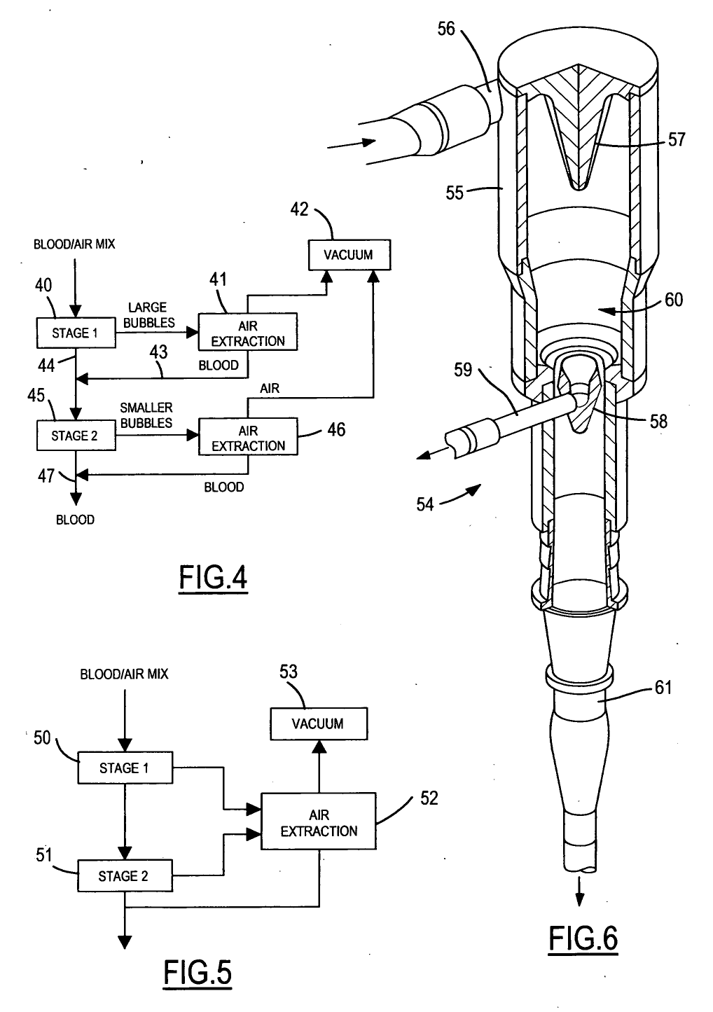

The overall air removal system of the invention may be implemented as shown in FIG. 4. To increase the effectiveness of air removal, multiple stages of voraxial devices are employed for capturing successively smaller air bubbles at each stage. Thus, a first stage 40 is sized to capture large bubbles (i.e., the chamber and bubble stop have diameters sufficiently large to separate and handle large bubbles). The pick-off flow with large air bubbles is provided to an air extraction unit 41 connected to a vacuum 42 via a valve (not shown) for drawing off air and providing a return blood flow 43 combining with a main blood outflow 44 of first stage 40. The combined blood flow which may still include small bubbles is provided to a second stage unit 45 which is sized to capture the smaller bubbles (i.e., having a smaller diameter than first stage 40). The diverted flow from second stage 45 is provided an air extraction unit 46 also connected to vacuum 42. Blood return from air extraction unit 46 is combined with blood in an air-reduced blood flow from second stage 45 to provide a combined blood output 47 which may be connected to a pump or other perfusion system components as shown in FIG. 1. The system in FIG. 4 minimizes blood contact with defoamers and other blood handling or air surfaces by maintaining a small prime volume in the first and second stages and by reducing the volume required for the air extraction units.

FIG. 5 shows an alternative embodiment wherein first stage 50 and second stage 51 share an air extraction unit 52. Air extraction unit 52 is connected to a vacuum source 53 and preferably passively operates an air removal valve as described in co-pending application Ser. No. 11/118,726. Flow through air extraction unit 52 is preferably driven by pressure differences in the perfusion system (i.e., each flow path must move from a higher to a lower pressure). In the embodiment of FIG. 5, flow through air extraction unit 52 is improved over the embodiment of FIG. 4 since a single return point following second stage 51 is utilized for the return blood flow. The return point may be within the chamber of a voraxial air separator device or may be provided at any downstream location in the blood flow path of the perfusion system.

FIG. 6 shows an alternative embodiment for a first stage air separator device that does not have a return line or return point for blood from the air extraction unit. Thus, a cylindrical housing 55 has a tangential input 56 at the top end along with a conical obstruction 57. A bubble stop 58 is connected to an air removal line 59 within a chamber 60. Blood flows around bubble stop 58 to exit at an outlet end 61 while an air-enhanced flow passes through bubble stop 58 and air removal line 59 to an air extraction unit (not shown).

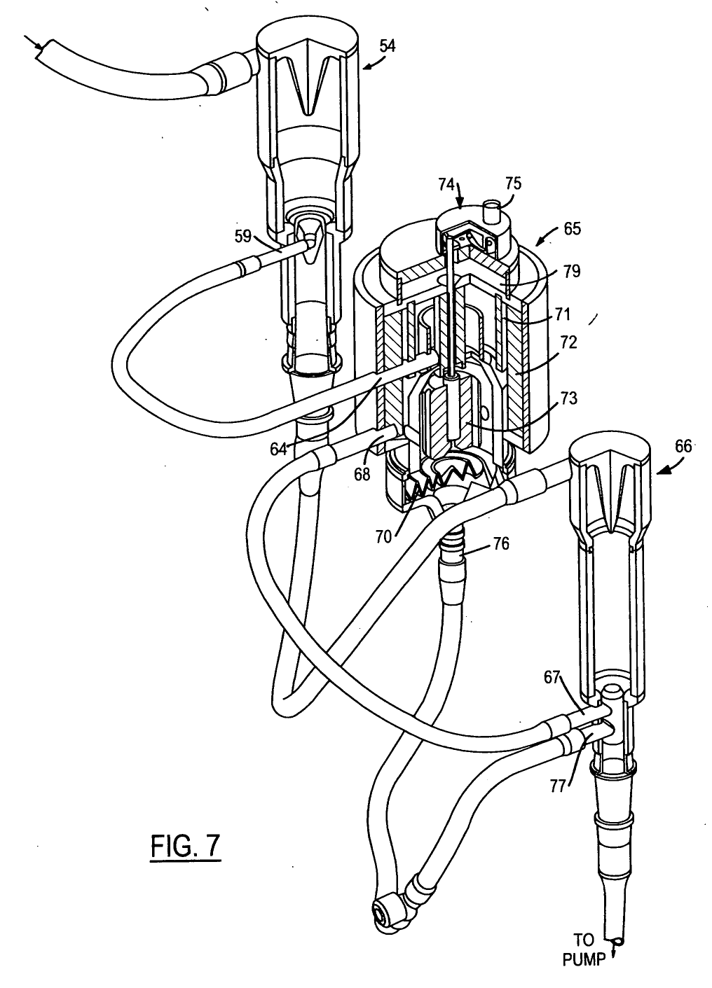

FIG. 7 shows an air removal system using a shared air extraction unit in greater detail. First voraxial stage 54 has its air removal line 59 connected to a first input 64 of an air extraction unit 65. A second stage voraxial air separation device 66 has an air removal line 67 connected to a second input 68 of air extraction unit 65. Air extraction unit 65 includes a screen 70 for blocking air bubbles and an internal chamber 71 for collecting air at the upper end thereof. Defoamer 72 is disposed within air extraction unit 65 to break up bubbles to allow air to collect in chamber 71. Collected air can move upward from chamber 71 through small holes (not shown) in a disk 79 to the vicinity of a valve 74. A float 73 drives valve 74 to selectively couple chamber 71 to a vacuum source through a vacuum port 75. Blood having substantially no bubbles remaining flows through an outlet 76 of air extraction unit 65 to a blood return line 77 of voraxial air separation device 66.

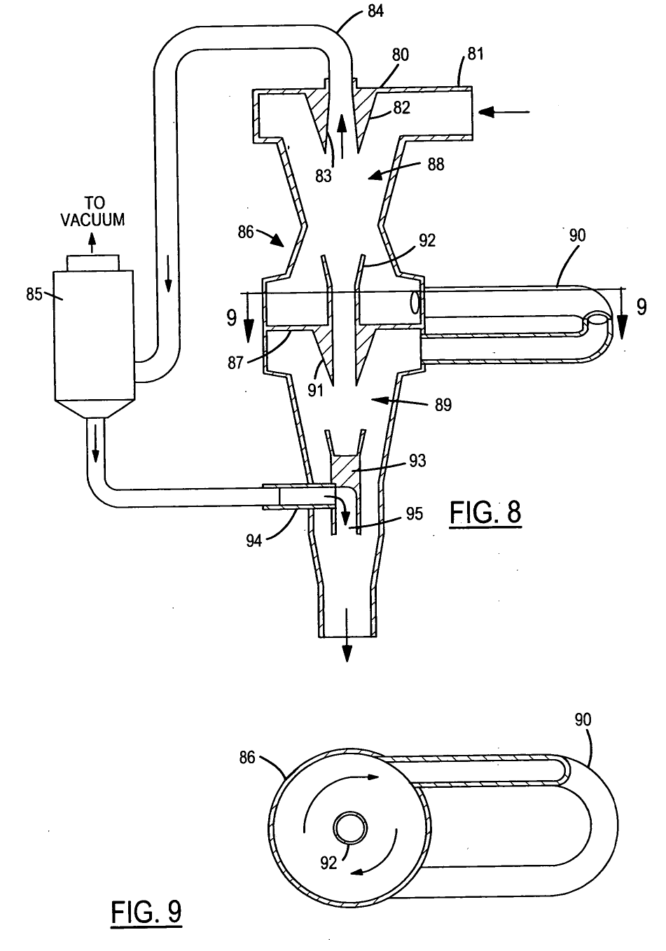

FIG. 8 shows an alternative embodiment wherein multiple stages of voraxial air separation are integrated into a single unit. The vortex flow of the present invention creates a column of air bubbles along the central longitudinal axis of the separation device. The previous embodiments have shown an air removal line proximate to the bubble stop at the downstream end of the column of bubbles. In FIG. 8, the pick-off point is shown proximate to the upstream end of the chamber and the bubble column so that air bubbles flow vertically along at least a portion of the longitudinal axis to reach the pick-off point.

A device 80 includes a tangential input 81 arranged proximate to a conical obstruction 82 to generate a vortex flow. Conical obstruction 82 has an internal passageway 83 connected to an air removal line 84 for conducting air bubbles along the central longitudinal axis to an air extraction unit 85. An intermediate vortex reinforcing section 86 includes a peripheral divider 87 separating the chamber within device 80 into an upper stage chamber 88 and a lower stage chamber 89. An arcuate loop 90 couples upper stage 88 to lower stage 89 and reestablishes a vortex flow by virtue of a conical surface 91 and a tangential reentry of loop 90. A common air bubble column is established using a throat body 92 connecting upper stage 88 and lower stage 89. With the shared axial air flow path between the stages, a single air return line 84 may be employed. Throat body 92 preferably has downward-facing conical surface 91 formed together therewith. A generally cup-shaped bubble stop 93 is provided at the downstream end of lower stage 89 in order to block the central longitudinal axis. A blood return line 94 is coupled to a return point 95 integrated with bubble stop 93 to reintroduce blood from air extraction unit 85 at the point of lowest pressure within device 80.

FIG. 9 shows a top horizontal cross section through the intermediate vortex reinforcing section 86. A vortex or spiral flow around throat body 92 exits tangentially into loop 90 from the upper stage and reenters tangentially into the lower stage. Thus, the strength of the vortex flow is maintained throughout the device at a level sufficient to segregate air from blood.

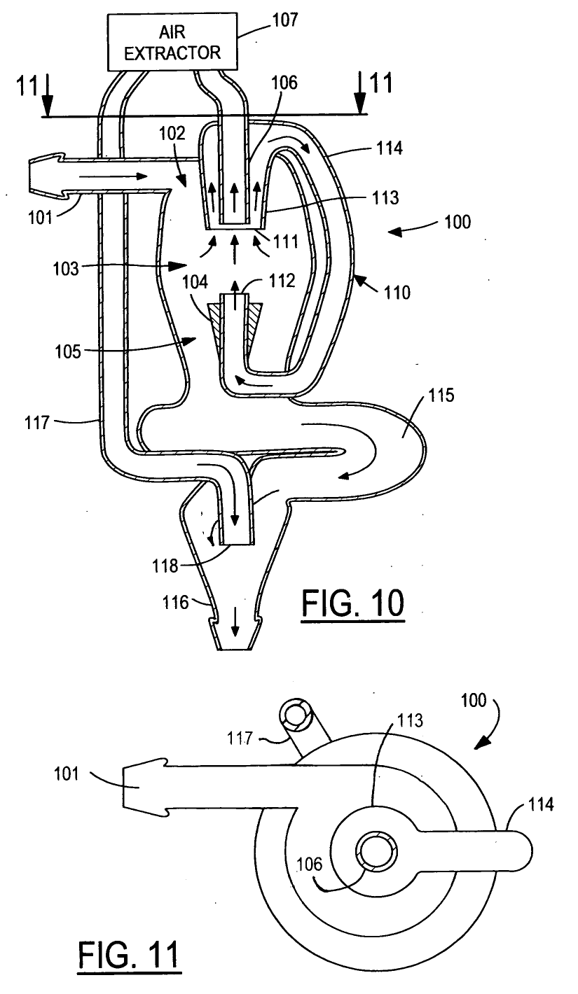

FIG. 10 shows yet another alternative embodiment for a voraxial air separator device 100 having a reduced overall height. An inlet 101 provides a tangential flow of blood/air mixture at an upper end 102 of a chamber 103. A bubble stop 104 is located at a lower end 105 of chamber 103. Air removal line 106 picks-off a column of air-enhanced flow and provides it to an air extractor 107.

The distance between the entrance to air removal line 106 and bubble stop 104 needs to provide sufficient time and area for migration of air bubbles from the annular flow of the vortex toward the central longitudinal axis. In order to shorten this distance, device 100 includes a recursive flow loop 110 having an inlet 111 spaced from the central longitudinal axis and having an outlet 112 aligned with the axis proximate to the bubble stop. Thus, an annular sleeve 113 is provided around return line 106 to create an upward flow annularly around return line 106 into a recursive flow tube 114 in order to transfer the annular flow portion direct to the central longitudinal axis, thereby reducing the radial distance over which all the bubbles have to migrate. Annular sleeve 113 preferably has an outer surface which is conical to help establish the vortex flow.

Air-reduced blood flow passing bubble stop 104 is guided through an exit volute 115 which reduces the velocity of the flow. Blood flowing through exit volute 115 is re-introduced tangentially to create a vortex around a blood return line 117. Blood return line 117 from air extractor unit 107 delivers return blood to a return point 118 located after exit volute 115. Return point 118 is at a lowered pressure due to the vortex around the end of return line 117. The lowered pressure helps drive flow through air extractor unit 107.

FIG. 11 is a top plan view of device 100. The top of annular sleeve 113 is disposed around air removal line 106 and is coupled with recursive tube 114 along the outside of device 100.

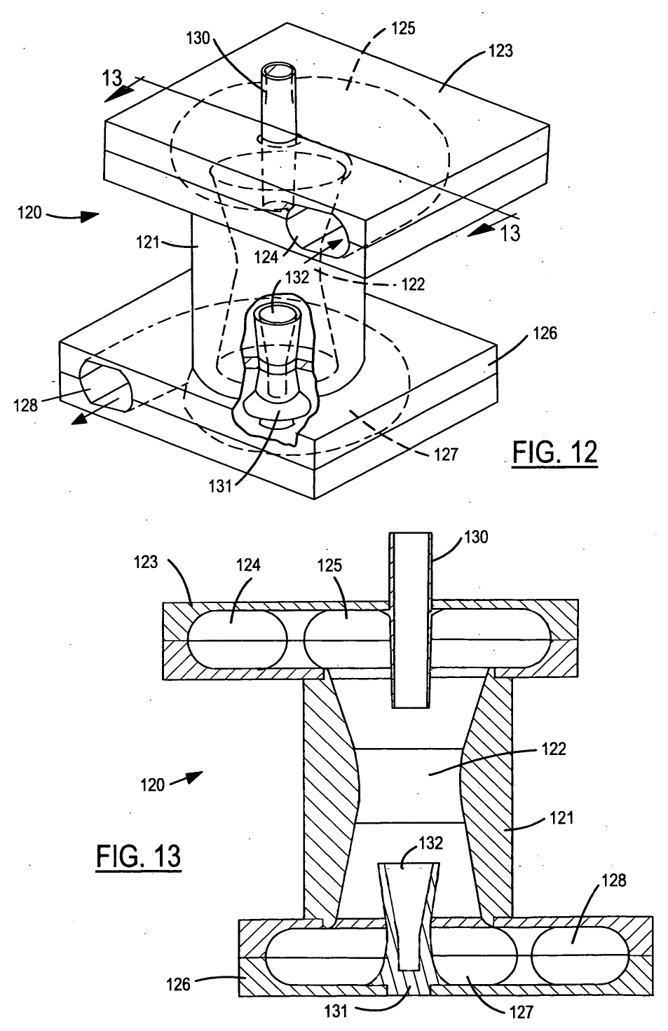

FIGS. 12 and 13 show an alternative embodiment of the air separator device adapted to remove micro-air bubbles and to have a very compact size. Device 120 includes a main body portion 121 having a generally cylindrical chamber 122. A vortex flow introducer block 123 is mounted atop body 121 and has a tangential input 124 leading to an entrance volute 125. An outlet block 126 is mounted to the bottom side of body 121 and has an exit volute leading to an outlet 128. Appropriate fittings (not shown) are attached to inlet 124 and outlet 128. Blocks 123 and 126 may be formed in two halves comprised of machined blocks or can be integrally molded. An air removal passageway 130 is mounted to block 123 and is aligned with the central longitudinal axis of chamber 122. A bubble stop 131 is mounted to block 126 and has an elongated cup 132 aligned with the central longitudinal axis. In a perfusion system, device 120 may preferably be used for separating residual micro-bubbles from blood in either a negative-pressure or a positive-pressure line.

1. An air removal system for removing air from blood flowing in a perfusion system, comprising:
a first elongated, substantially cylindrical chamber having a central longitudinal axis between an upstream end and a downstream end, wherein said chamber provides a substantially unobstructed blood flow path therethrough along a wall of said chamber, and wherein said downstream end provides an air-reduced blood flow to said perfusion system;
a vortex flow introducer substantially located at said upstream end;
a bubble stop blocking said central longitudinal axis prior to said downstream end;
an air removal line coupled to said central longitudinal axis between said upstream end and said bubble stop;
an air extraction unit having an input coupled to said air removal line, an air output for coupling to a vacuum source via a valve, and a blood output; and
a blood return line coupled to said blood output for returning blood passing through said air extraction unit to a return point that is downstream of said bubble stop.

2. The system of claim 1 wherein said vortex flow introducer comprises a tangential input port directing inflowing blood tangentially into said chamber.

3. The system of claim 1 wherein said vortex flow introducer comprises a downward-facing conical wall disposed around said central longitudinal axis.

4. The system of claim 1 wherein said air removal line is connected to a pick-off point proximate to said upstream end, whereby air flows vertically along at least a portion of said central longitudinal axis to reach said pick-off point.

5. The system of claim 4 wherein said bubble stop comprises a blocking wall substantially perpendicular to said central longitudinal axis.

6. The system of claim 5 wherein said return point is located beneath said blocking wall.

7. The system of claim 4 further comprising a recursive flow loop having an inlet spaced from said central longitudinal axis and having an outlet aligned with said central longitudinal axis.

8. The system of claim 7 wherein said inlet is comprised of an annular sleeve spaced around said air removal line and having a conical outer surface.

9. The system of claim 1 wherein said air removal line is connected to a pick-off point proximate to said bubble stop.

10. The system of claim 9 wherein said bubble stop and said pick-off point are integrally formed into an elbow.

11. The system of claim 1 further comprising:
an exit volute downstream of said bubble stop for reducing vorticity of said air-reduced blood.

12. The system of claim 1 further comprising:
an intermediate vortex reinforcing section including a peripheral divider separating said chamber into upper and lower stages and a loop for removing blood at an exit from said upper stage and following an arcuate path to reintroduce said blood tangentially at an entrance to said lower stage.

13. The system of claim 12 wherein said intermediate vortex reinforcing section further includes a throat body providing a passage along said central longitudinal axis between said upper and lower stages to provide a shared axial air flow path for said upper and lower stages.

14. The system of claim 13 wherein said throat body has a downward-facing conical outer surface in said lower stage.

15. The system of claim 1 wherein said return point is located within said chamber.

16. The system of claim 1 further comprising:
a second stage cylindrical chamber connected by tubing with said downstream end of said first cylindrical chamber;
a second stage vortex flow introducer in said second stage cylindrical chamber;
a second stage bubble stop; and
a second stage air removal line coupled to said air extraction unit;
wherein said return point is downstream of said second stage bubble stop.

17. The system of claim 16 wherein said second stage cylindrical chamber has a smaller dimension than said first cylindrical chamber adapted to capture smaller air bubbles.

18. The system of claim 1 wherein said air extraction unit includes a separating media in a flow path of said air extraction unit for contacting said blood.

19. The system of claim 18 wherein said separating media comprises a screen.

20. The system of claim 18 wherein said separating media comprises a blood defoamer.

21. An air separator device for removing air from flowing blood, comprising:
a first elongated, substantially cylindrical chamber having a central longitudinal axis between an upstream end and a downstream end, wherein said chamber provides a substantially unobstructed blood flow path therethrough along a wall of said chamber, and wherein said downstream end provides an air-reduced blood flow;
a vortex flow introducer substantially located at said upstream end;
a bubble stop blocking said central longitudinal axis remote from said upstream end; and
an air removal passageway coupled to said central longitudinal axis proximate to said upstream end to receive a bubble flow moving vertically upward above said bubble stop.

22. The device of claim 21 further comprising a recursive flow loop having an inlet spaced from said central longitudinal axis and having an outlet aligned with said central longitudinal axis.

23. The device of claim 22 wherein said inlet is comprised of an annular sleeve spaced around said air removal passageway and having a conical outer surface.

24. The system of claim 21 further comprising:
an exit volute downstream of said bubble stop for reducing velocity of said air-reduced blood.

25. The device of claim 21 further comprising:
an intermediate vortex reinforcing section including a peripheral divider separating said chamber into upper and lower stages and a loop for removing blood at an exit from said upper stage and following an arcuate path to reintroduce said blood tangentially at an entrance to said lower stage.

26. The device of claim 25 wherein said intermediate vortex reinforcing section further includes a throat body providing a passage along said central longitudinal axis between said upper and lower stages to provide a shared axial air flow path for said upper and lower stages.

27. The device of claim 21 wherein said vortex flow introducer comprises a tangential input port directing inflowing blood tangentially into said chamber.

28. The device of claim 21 wherein said vortex flow introducer comprises a downward-facing conical wall disposed around said central longitudinal axis.

29. A method of removing air from blood flowing in a perfusion system comprising the steps of:
generating a vortex flow at an upstream end of a chamber and into an intermediate section of said chamber;
segregating air from blood along a central longitudinal axis in said intermediate section;
blocking downward flow along said central longitudinal axis at a bubble stop location below said intermediate section;
outputting an air-reduced blood flow from a downstream end of said chamber, said air-reduced blood flow comprising a portion of said vortex flow that is not blocked at said bubble stop;
generating an extraction flow between said central longitudinal axis and an air extraction unit outside of said chamber;
evacuating air from said extraction flow to a vacuum source through a valve; and
returning blood from said extraction flow to said perfusion system.

30. The method of claim 29 wherein said extraction flow is obtained from an air pick-off point proximate to said bubble stop location.

31. The method of claim 29 wherein said extraction flow is obtained from an air pick-off point proximate to said upstream end of said chamber on said central longitudinal axis.

32. The method of claim 31 further comprising the step of:
generating a recursive flow from an annulus around said central longitudinal axis to a recursive outlet proximate to said bubble stop location on said central longitudinal axis.

33. The method of claim 31 wherein said vortex flow is created in upper and lower stages connected by a vortex reinforcing loop.

34. The method of claim 33 wherein a throat body provides a passage along said central longitudinal axis between said upper and lower stages to provide a shared axial air flow path for said upper and lower stages.

35. The method of claim 29 further comprising the steps of:
generating a second vortex flow at an upstream end of a second chamber and into a second intermediate section of said second chamber;
segregating air from blood along a second central longitudinal axis in said second intermediate section;
blocking downward flow along said second central longitudinal axis at a second bubble stop location below said second intermediate section;
outputting a second air-reduced blood flow from a downstream end of said second chamber, said second air-reduced blood flow comprising a portion of said second vortex flow that is not blocked at said second bubble stop; and
generating a second extraction flow between said second central longitudinal axis and said air extraction unit outside of said second chamber.

36. The method of claim 35 wherein said second chamber has a smaller volume than a volume of said chamber.
